(19) United States
(12) Patent Application Publication
(10) Pub. No.: US 2022/0096598 A1
(43) Pub. Date: Mar. 31, 2022

(54) FIBROBLAST GROWTH FACTOR 21 VARIANT, AND FUSION PROTEIN AND USE THEREOF

(71) Applicants: Haifeng DUAN, Beijing (CN); BEIJING SHUANGYIN BIOTECHNOLOGY CO., LTD., Beijing (CN)

(72) Inventors: Haifeng DUAN, Beijing (CN); Jing XIE, Beijing (CN); Jingbo GONG, Beijing (CN); Binghua XUE, Beijing (CN); Xiuxiao XIAO, Beijing (CN); Qunwei ZHANG, Beijing (CN); Meilan CUI, Beijing (CN); Rumeng PANG, Beijing (CN); Tingting YU, Beijing (CN); Rui WANG, Beijing (CN)

(21) Appl. No.: 17/310,377

(22) PCT Filed: Nov. 22, 2019
(86) PCT No.: PCT/CN2019/120357

§ 371 Date: Jul. 29, 2021

(30) Foreign Application Priority Data
Jan. 30, 2019 (CN) 201910093697.4

(51) Int. Cl. A61K 38/18; A61P 9/10; C12N 15/85

(52) U.S. Cl. CPC A61K 38/1825; C12N 15/85; A61P 9/10

(57) ABSTRACT

It discloses a fibroblast growth factor 21 variant, a fusion protein comprising such fibroblast growth factor 21 variant, a GLP-1 variant and a FC sequence, and a use thereof. The fusion protein of the present invention has high activity, long half-life and a novel structure, and can significantly decrease blood sugar, body weight, and improve fat metabolism. The present invention also provides a fusion gene, an expression construct, and a host cell comprising an encoding nucleotide sequence of the fusion protein, and a use of the fusion protein, the fusion gene, the expression construct, the host cell, and the pharmaceutical composition in the preparation of drugs for treating obesity, hyperlipidemia, diabetes, and cardiovascular and cerebrovascular diseases.

### TECHNICAL FIELD

The present invention relates to the field of biopharmaceuticals.

Specifically, the present invention relates to a fibroblast growth factor 21 variant, more specifically, relates to a fusion protein containing such fibroblast growth factor 21 variant, a GLP-1 variant and a FC sequence, and a use thereof.

### BACKGROUND ART

The sedentary lifestyle and excessive calorie intake of modern people are exacerbating the globe epidemic of obesity, non-alcoholic fatty liver and type 2 diabetes. Such defects on energy metabolism can further induce severe cardiovascular diseases or even tumors. However, currently, effective treatments for obesity and related complications are very limited, and thus, there is an urgent need for a new drug that has less side effects and can correct the imbalance of energy metabolism.

Fibroblast growth factor 21 (FGF21) is a member of the fibroblast growth factor (FGF) family. It is an important metabolic regulator that is involved in regulating the balance between energy and glucolipid metabolism by activating FGF receptors (FGFRs) and co-receptor β-klotho (KLB) of the tyrosine kinase transmembrane receptor family (Sonoda J, Chen M Z, Baruch A. Hormone Molecular Biology and Clinical Investigation, 2017, 30(2):1-13). The wild-type human FGF21 is a secreted polypeptide containing 181 amino acids, which has an amino acid sequence homology with mouse FGF21 of 81%. N-terminus of human FGF21 sequence is involved in the interaction with FGFRs, meanwhile C-terminus is essential for binding the co-receptor KLB (Micanovic R, Raches D W, Dunbar J D, etc. Journal of Cellular Physiology, 2009, 219(2):227-234). FGF21 can relieve hyperglycemia, reduce triglyceride levels and improve lipid metabolism mainly by activating AMPK/SIRT1/PGC1α (Chau M D, Gao J, Yang Q, etc. Proceedings of the National Academy of Sciences USA, 2010, 107(28):12553-12558). FGF21 is considered to be an effective target for the treatment of various metabolic diseases. For example, by injecting recombinant FGF21 protein into mice and subjects, serum glucose, triglyceride and cholesterol levels can be reduced, insulin sensitivity can be increased, energy metabolism can be promoted, and fatty liver and obesity can be relieved (Hecht R, Li Y S, Sun J et al. PLoS One, 2012, 7(11): e49345; Kharitonenkov A, Beals J M, Micanovic R, et al. PLoS One, 2013, 8(3): e58575). The half-life of FGF21 in the body is very short, and in primates, it's only 0.5-2h. Moreover, in the blood, FGF21 is tended to be cleaved by protease DPPIV at P2 and P4 sites on N-terminus and cleaved by fibroblast activation protein (FAP) at P171 site on C-terminus, thereby losing its activity (Sonoda J, Chen M Z, Baruch A. Hormone Molecular Biology and Clinical Investigation, 2017, 30(2):1-13). These problems have become huge challenges in the development of FGF21 as a drug for the treatment of metabolic diseases.

Glucagon-like peptide-1 (GLP-1) is a member of the glucagon peptide family, an endogenous incretin, involved in the process of glucose transport and metabolism (Lee S, Lee D Y. Annals of Pediatric Endocrinology & Metabolism, 2017, 22(1):15-26). There are two forms of GLP-1 in the human body, i.e., GLP-1 (7-36) mainly secreted by pancreatic tissue, and GLP-1 (7-37) mainly secreted by the intestine. GLP-1 can activate the downstream cAMP-dependent signaling pathway by activating GLP-1 receptor (GLP-1R) of G protein-coupled receptor family. GLP-1 receptor agonists are also currently attractive targets for the treatment of type 2 diabetes, and a variety of drugs, such as Novo Nordisk's liraglutide and Eli Lilly's Dulaglutide, have been approved for clinical use in the treatment of type 2 diabetes. These GLP-1 receptor agonist drugs also have the effect of losing weight, which is, however, mainly achieved by suppressing the appetite and controlling the food intake, thereby greatly reducing the patient's quality of life (Glaesner W, Vick A M, Millican R, etc. Diabetes/Metabolism Research and Review, 2010, 26(4): 287-296).

Although a considerable progress of the research of fusion protein has been made in the past few years, and the glorious prospect of its ultimate clinical application can be expected, generally, when directly prepared based on the wild-type protein sequence, the spatial structure of the fusion protein will be affected, and therefore, its activity will be affected. The patent application CN201280 057819.0 has disclosed a novel protein containing fibroblast growth factor (FGF21) and other metabolic regulators known to improve the metabolic profile of the subject, including its variants. Also disclosed are methods for the treatment of FGF21 related diseases, GLP-1 related diseases and Exendin-4 related diseases, including metabolic conditions. However, the fusion protein obtained in this publication has no activity high enough, it has to be administered frequently in actual clinical use, and its clinical compliance is needed to be further improved.

Therefore, there is still a need for therapeutic agents for FGF21-related diseases with higher activity and better compliance.

### SUMMARY OF THE INVENTION

In view of the above-mentioned problems of the prior art, the present invention provides a fusion protein with GLP-1 and FGF21 activities, and a method for preparing the same and a use thereof as well. Also provided is a use of the protein according to the present invention for treating or preventing metabolic diseases including obesity, hyperlipidemia, diabetes, and cardiovascular and cerebrovascular diseases. Compared with the prior art, the fusion protein according to the present invention has higher activity, longer half-life and a novel structure, and can significantly reduce blood sugar, blood fat, body weight and improve fat metabolism.

Specifically, the object of the present invention is to provide the following aspects.

In one aspect, the present invention provides a human fibroblast growth factor 21 (FGF21) variant having an amino acid sequence as shown in the following general formula I:

General formula I
DSSPLLQFGGQVRQX₁₅YLYTDDAQQTEAHLEIREDGTVGGAADQSPESL
LQLKALKPGVIQILGVKTSRFLCQRPDGALYGSLHFDPEACSFREX₉₄LL
EDGYNVYQSEAHGLPLHX₁₁₄PGNKSPHRDPAPRGPX₁₃₀RFLPLPGLPPA
LPEPPGILAPQPPDVGSSDPLSMVGGSQGRSPSYX₁₇₆S

wherein,

X₁₅ is Arg or Val, X₉₄ is Leu or Arg, X₁₁₄ is Leu or Cys, X₁₃₀ is Ala or Cys, and X₁₇₆ is Ala or Glu;

one and only one of X₉₄ and X₁₁₄ is Leu, and at most one of X₉₄ and X₁₁₄ is Ala;

and preferably, the amino acid sequence of the variant is as shown in any one of SEQ ID NO:1-4.

In another aspect, the present invention provides a fusion protein represented by the following general formula:

G-L-Fc-L-F, or G-L-G-L-Fc-L-F;

wherein,

F represents the human FGF21 variant according to the present invention;

G represents a GLP-1 variant (GLP-lv) having an amino acid sequence shown in SEQ ID NO:5;

L represents a linker sequence;

FC represents human or animal immunoglobulin and its subtypes and variants, human or animal albumin and its variants, or PEG.

In the fusion protein according to the present invention, the general formula of L is (GGGGS)n, wherein n is an integer from 0 to 5, preferably is 3. FC preferably represents an IgG4FC fragment, and more preferably contains the amino acid sequence shown in SEQ ID NO:17.

According to the present invention, the fusion protein further contains other antigens, functional amino acid sequences and/or signal peptide sequences. Preferably, the functional amino acid sequences are histidine tags or GST tags. Preferably, the amino acid sequence of the fusion protein is as shown in any one of SEQ ID NO: 6-9, 18, or 24-26.

In yet another aspect, the present invention provides a fusion gene, containing the coding nucleotide sequence of the human FGF21 variant or the fusion protein according to the present invention. The coding nucleotide sequence of the FGF21 variant is as shown in any one of SEQ ID NO:20-23. The coding nucleotide sequence of the fusion protein is as shown in any one of SEQ ID NO:10-13, 19, or 27-29.

In still another aspect, the present invention provides an expression construct, containing the coding nucleotide sequence of the human FGF21 variant or the fusion protein according to the present invention.

The expression construct according to the present invention is a prokaryotic expression construct, which is preferably a pET vector system.

Alternatively, the expression construct according to the present invention is an eukaryotic expression construct, which is preferably a plasmid DNA vector, preferably pVAX1 vector and pSV1.0 vector; a recombinant viral vector, preferably recombinant vaccinia virus vector, recombinant adenovirus vector, or recombinant adeno-associated virus vector; or a retroviral vector, preferably HIV virus vector, or lentiviral vector.

In still another aspect, the present invention provides a host cell, containing the expression construct according to the present invention. Preferably, when the expression construct is a prokaryotic expression construct, the host cell is a prokaryotic cell, preferably bacterial cell; alternatively, when the expression construct is a eukaryotic expression construct, the host cell is a eukaryotic cell, preferably mammalian cell, more preferably CHO cell.

In still another aspect, the present invention provides a pharmaceutical composition, comprising the human FGF21 variant or the fusion protein according to the present invention.

In still another aspect, the present invention provides a method for preparing a human FGF21 variant or a fusion protein, comprising the step of cloning the coding nucleotide sequence of the fusion protein into an expression vector.

Preferably, the method comprises the steps as follows:
1) constructing the nucleotide sequence of the human fibroblast growth factor 21 variant or the fusion protein;
2) constructing an expression vector containing the nucleotide sequence of step 1);
3) utilizing the expression vector of step 2) to transfect or transform a host cell and allow the nucleotide sequence to be expressed in the host cell;
4) purifying the protein expressed in step 3);

and more preferably, in step 3), the host cell is a CHO-S cell.

The present invention also provides a use of the above-mentioned human FGF21 variant, the fusion protein, the fusion gene, the expression construct, the host cell, or the pharmaceutical composition in the preparation of drugs for obesity, hyperlipidemia, diabetes, and cardiovascular and cerebrovascular diseases.

The amino acid sequences of the human FGF21 variants (FGF21v) Fv2, Fv3, Fv4 and Fv5 of the present invention are shown in SEQ ID NOs: 1, 2, 3 and 4:

Fv2
SEQ ID NO: 1
DSSPLLQFGGQVRQRYLYTDDAQQTEAHLEIREDGTVGGAADQSPESLLQ
LKALKPGVIQILGVKTSRFLCQRPDGALYGSLHFDPEACSFRELLLEDGY
NVYQSEAHGLPLHCPGNKSPHRDPAPRGPCRFLPLPGLPPALPEPPGILA
PQPPDVGSSDPLSMVGGSQGRSPSYAS
Fv3
SEQ ID NO: 2
DSSPLLQFGGQVRQVYLYTDDAQQTEAHLEIREDGTVGGAADQSPESLLQ
LKALKPGVIQILGVKTSRFLCQRPDGALYGSLHFDPEACSFRELLLEDGY
NVYQSEAHGLPLHCPGNKSPHRDPAPRGPCRFLPLPGLPPALPEPPGILA
PQPPDVGSSDPLSMVGGSQGRSPSYAS
Fv4
SEQ ID NO: 3
DSSPLLQFGGQVRQVYLYTDDAQQTEAHLEIREDGTVGGAADQSPESLLQ
LKALKPGVIQILGVKTSRFLCQRPDGALYGSLHFDPEACSFRERLLEDGY
NVYQSEAHGLPLHLPGNKSPHRDPAPRGPARFLPLPGLPPALPEPPGILA
PQPPDVGSSDPLSMVGGSQGRSPSYES
Fv5
SEQ ID NO: 4
DSSPLLQFGGQVRQVYLYTDDAQQTEAHLEIREDGTVGGAADQSPESLLQ
LKALKPGVIQILGVKTSRFLCQRPDGALYGSLHFDPEACSFRELLLEDGY
NVYQSEAHGLPLHCPGNKSPHRDPAPRGPCRFLPLPGLPPALPEPPGILA
PQPPDVGSSDPLSMVGGSQGRSPSYES

The nucleotide sequences of the human FGF21 variants (FGF21v) Fv2, Fv3, Fv4 and Fv5 of the present invention are shown in SEQ ID NOs: 20, 21, 22 and 23:

Compared with the prior art, the present invention has the following advantages:

In the embodiments of the present invention, the activity of the fusion protein according to the present invention was evaluated, utilizing a normal mouse glucose load model of low-density lipoprotein-deficient mice and taking dulaglutide as a positive control drug. The results showed that the fusion protein according to the present invention has a good curative effect and obvious advantages in the treatment of hyperlipidemia.

### DESCRIPTION OF FIGURES

Hereinafter, the embodiments of the present invention will be described in detail with reference to the drawings, in which:

FIG. 1 shows a plasmid map of pcDNA3.4-fusion protein;

FIG. 2 shows the effect of wild-type GF protein and its mutants on the phosphorylation of AMPK and total AMPK in HepG2 cells; con means cells without drug treatment; * represents the significant difference compared with con (p<0.05); ** represents the extremely significant difference compared with con (p<0.001); ## represents the extremely significant difference compared with GF (p<0.001);

FIG. 3 shows the effect of different proteins on the expression of luciferase in HEK293-GLP1R/β-klotho/CRE-Luciferase cells; (A) the comparison of EC50 values of different GGFvn proteins with corresponding GFvn proteins, n=2-5; (B) the comparison of EC50 values among G, GFv5 and GGFv5;

FIG. 4 shows the effect of GFv5 and GGFv5 on the body weight (A) and food intake (B) of ld1r^{-/-} mice; * represents the significant difference compared with con group (p<0.05); # represents the significant difference compared with G group (p<0.05); $ represents the significant difference compared with GFv5 (p<0.05);

FIG. 5 shows the effect of GFv5 and GGFv5 on blood lipids of ld1r^{-/-} mice; * represents the significant difference compared with con group (p<0.05); # represents the significant difference compared with G group (p<0.05); $ represents the significant difference compared with GFv5 (p<0.05).

### EMBODIMENTS

The present invention will be further described in detail below through the embodiments and examples. Through these descriptions, the characteristics and advantages of the present invention will become clearer.

The term “exemplary” herein means “serving as an example, embodiment, or illustration.” Any embodiment described herein as “exemplary” need not be construed as being superior to or better than other embodiments.

Unless otherwise specified, the reagents used in the following examples are analytical grade reagents, and are commercially available.

Example 1 Preparation of the Fusion Protein of the Present Invention

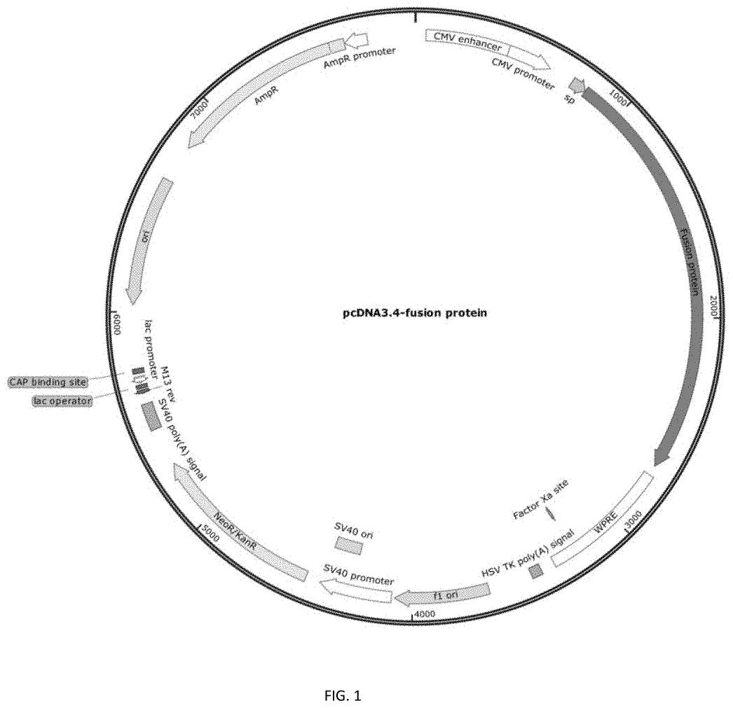

The fusion protein was prepared by the conventional technical means of the present invention, specifically including the following step of: utilizing pcDNA3.4-TOPO TA cloning kit (purchased from Invitech (Shanghai) Trading Co., Ltd.) to construct pcDNA3.4 plasmid containing the fusion protein (the plasmid map was shown in FIG. 1). This plasmid was used to transfect ExpiCHO-S cells, and the ExpiCHO expression system (purchased from Invitech (Shanghai) Trading Co., Ltd.) was used to express the protein.

The fusion protein according to the present invention can be obtained after purificated by the method as described below: the supernatant was filtered with a 0.22 μm membrane to remove cell debris; protein A affinity column HiTrap MabSelect SuRe (purchased from General Electric Company) was treated with 5 column volumes of equilibration buffer (5.6 mM NaH₂PO₄, 14.4 mM Na₂HPO₄, 0.15M NaCl, pH7.2), and then the supernatant was loaded; after loading, the poorly bound contaminated proteins were washed off to the baseline with buffer (5.6 mM NaH₂PO₄.H₂O, 14.4 mM Na₂HPO₄, 0.5 M NaCl, pH7.2); the protein was eluted with the eluent of 50 mM citric acid/sodium citrate buffer (containing 0.02% Tween-80+5% mannitol, pH3.2), and then was adjusted to pH7.0 by using 1M Tris-Cl (pH8.0). The purified sample was filtered through a 0.22 μm membrane and sterilized, and then stored at 4° C.

Specifically, the fusion protein according to the present invention can be represented by the general formulas of GL-Fc-L-Fv2, GL-Fc-L-Fv3, GL-Fc-L-Fv4, and GL-Fc-L-Fv5, the amino acid sequences were as shown in SEQ ID NO:6-9, respectively, and the nucleotide sequences were as shown in SEQ ID NO:10-13, respectively.

G-L-Fc-L-Fv2 SEQ ID NO:6 (the part in bold represents the amino acid sequence of the GLP-1 variant, the part in bold italics represents the amino acid sequence of the linker sequence, and the part underlined represents the amino acid sequence of Fc):

HGEGTFTSDVSSYLEEQAAKEFIAWLVKGGG [symbol] AESK
YGPPCPPCPAPEAAGGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLG [symbol] DSSPLLQFGG
QVRQRYLYTDDAQQTEAHLEIREDGTVGGAADQSPESLLQLKALKPGVIQ
ILGVKTSRFLCQRPDGALYGSLHFDPEACSFRELLLEDGYNVYQSEAHGL
PLHCPGNKSPHRDPAPRGPCRFLPLPGLPPALPEPPGILAPQPPDVGSSD
PLSMVGGSQGRSPSYAS

G-L-Fc-L-Fv3 SEQ ID NO:7 (the part in bold represents the amino acid sequence of the GLP-1 variant, the part in bold italics represents the amino acid sequence of the linker sequence, and the part underlined represents the amino acid sequence of Fc):

HGEGTFTSDVSSYLEEQAAKEFIAWLVKGGG [symbol] AES
KYGPPCPPCPAPEAAGGPSVFLFPPKPKDTLMISRTPEVTCVVVDVSQE
DPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTC
LVKGFYPSDIAVEWESNGQPENNYKTTPPVLDSDGSFFLYSRLTVDKSR
WQEGNVFSCSVMHEALHNHYTQKSLSLSLG [symbol] DSSP
LLQFGGQVRQVYLYTDDAQQTEAHLEIREDGTVGGAADQSPESLLQLKA
LKPGVIQILGVKTSRFLCQRPDGALYGSLHFDPEACSFRELLLEDGYNV
YQSEAHGLPLHCPGNKSPHRDPAPRGPCRFLPLPGLPPALPEPPGILAP
QPPDVGSSDPLSMVGGSQGRSPSYAS

G-L-Fc-L-Fv4 SEQ ID NO:8 (the part in bold represents the amino acid sequence of the GLP-1 variant, the part in bold italics represents the amino acid sequence of the linker sequence, and the part underlined represents the amino acid sequence of Fc):

HGEGTFTSDVSSYLEEQAAKEFIAWLVKGGG [symbol] AESK
YGPPCPPCPAPEAAGGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLG [symbol] DSSPLLQFGG
QVRQVYLYTDDAQQTEAHLEIREDGTVGGAADQSPESLLQLKALKPGVIQ
ILGVKTSRFLCQRPDGALYGSLHFDPEACSFRERLLEDGYNVYQSEAHGL
PLHLPGNKSPHRDPAPRGPARFLPLPGLPPALPEPPGILAPQPPDVGSSD
PLSMVGGSQGRSPSYES

G-L-Fc-L-Fv5 SEQ ID NO:9 (the part in bold represents the amino acid sequence of the GLP-1 variant, the part in bold italics represents the amino acid sequence of the linker sequence, and the part underlined represents the amino acid sequence of Fc):

HGEGTFTSDVSSYLEEQAAKEFIAWLVKGGG [symbol] AESK
YGPPCPPCPAPEAAGGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLG [symbol] DSSPLLQFGG
QVRQVYLYTDDAQQTEAHLEIREDGTVGGAADQSPESLLQLKALKPGVIQ
ILGVKTSRFLCQRPDGALYGSLHFDPEACSFRELLLEDGYNVYQSEAHGL
PLHCPGNKSPHRDPAPRGPCRFLPLPGLPPALPEPPGILAPQPPDVGSSD
PLSMVGGSQGRSPSYES

Additionally, the inventors prepared a wild-type G-L-Fc-L-F fusion protein, having an amino acid sequence as shown in SEQ ID NO:14:

HGEGTFTSDVSSYLEEQAAKEFIAWLVKGGGGGGGSGGGGSGGGGSAESK
YGPPCPPCPAPEAAGGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGN
VFSCSVMHEALHNHYTQKSLSLSLGGGGGSGGGGSGGGGSHPIPDSSPLL
QFGGQVRQRYLYTDDAQQTEAHLEIREDGTVGGAADQSPESLLQLKALKP
GVIQILGVKTSRFLCQRPDGALYGSLHFDPEACSFRELLLEDGYNVYQSE
AHGLPLHLPGNKSPHRDPAPRGPARFLPLPGLPPALPEPPGILAPQPPDV
GSSDPLSMVGPSQGRSPSYAS

The nucleotide sequence of said fusion protein was as shown in SEQ ID NO:15.

The nucleotide sequence of the signal peptide used in said fusion protein was shown in SEQ ID NO:16:

ATGCCGTCTTCTGTCTCGTGGGGCATCCTCCTGCTGGCAGGCCTGTGCTG
CCTGGTCCCTGTCTCCCTGGCT

Example 2 Effect of the Fusion Protein According to the Present Invention on the AMPK Signal Pathway of HepG2 Cells

HepG2 cells (obtained from the Academy of Military Medical Sciences) were cultured to a confluence of more than 90% by using DMEM medium containing 10% FBS, and then were digested and resuspended to be inoculated into a 6-well plate according to 2.5×10⁵ cells per well. Then, 2 mL of DMEM medium containing 10% FBS was added into each well to culture the cells overnight at 37° C. and 5% CO₂ saturated humidity up to 70%-80% saturation. Subsequently, the original medium was removed and replaced by a fresh pre-warmed serum-free DMEM medium. After cultured for another 6 hours, 100 nM purified wild-type fusion protein G-L-Fc-L-F(GF) and its four mutants G-L-Fc-L-Fv2(GFv2), G-L-Fc-L-Fv3(GFv3), G-L-Fc-L-Fv4(GFv4), G-L-Fc-L-Fv5(GFv5) were added. After treated for 24 hours, the culture supernatant was removed, and the cells were digested and collected. Then, the cells were washed once with pre-cooled PBS, and lysed by using RIPA lysis buffer containing 1% PMSF (purchased from Beijing Kangwei Century Biotechnology Co., Ltd.) to extract total protein according to the instruction. 15 μL of total protein was taken to detect the expression levels of total AMPK (AMPKα antibody) and phosphorylated AMPK (pAMPK, phospho-AMPKα (Thr172) antibody) in the cells (both antibodies were purchased from Cell Signaling Technologies) by immunoblotting.

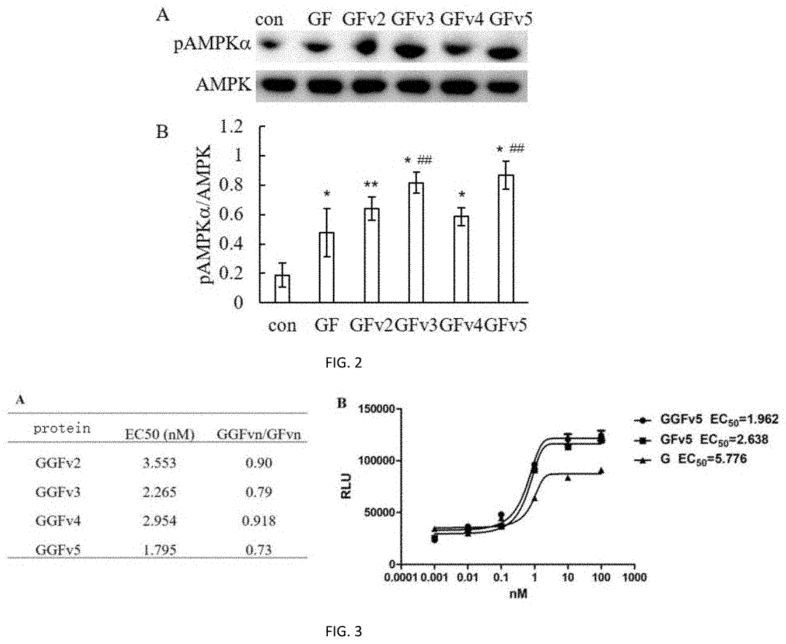

The results were shown in FIG. 2. After treated with the wild-type GF fusion protein and four GF mutants, the phosphorylation level of AMPK in HepG2 cells was significantly higher than that of the control group (con) (increased ratio of pAMPK/AMPK), indicating that all the proteins were active. Particularly, after treated with mutants GFv3 and GFv5, the phosphorylation level of AMPK in HepG2 cells was significantly higher than that treated with GF protein, indicating that the activity of these two mutant proteins was higher than that of the wild-type protein.

Example 3 Comparison of the Activation Effects of GF Fusion Protein and its Mutants on GLP1 Receptor and FGF21 Receptor

The HEK293 cells (HEK293-GLP1R/β-klotho/CRE-Luciferase) expressing GLP1 receptor, FGF21 co-receptor (β-klotho) and CRE-luciferase inducible expression system were cultured to a confluence of more than 90% by using DMEM medium containing 10% FBS, and then were digested and resuspended to be inoculated into a 96-well plate according to 4×104 cells per well. Then, 100 μL of DMEM medium containing 10% FBS was added into each well to culture the cells overnight at 37° C. and 5% CO₂ saturated humidity. On the second day, a wild-type fusion protein G-L-Fc-L-F(GF) and its four mutants G-L-Fc-L-Fv2(GFv2), G-L-Fc-L-Fv3(GFv3), G-L-Fc-L-Fv4(GFv4), G-L-Fc-L-Fv5(GFv5) with different concentration gradients (0, 0.001, 0.01, 0.1, 1, 10, 100 nM) were added. After treated for 6-8h, the culture supernatant was removed, and the cells were washed twice with PBS and lysed according to the instructions to detect the expression of luciferase (using a single luciferase reporter gene detection kit, purchased from Beijing Yuanpinghao Biotechnology Co., Ltd.). By analyzing the data with a software Graphpad Prism, EC₅₀ value of a GF protein was obtained and as shown in Table 1. The results showed that EC₅₀ values of the four mutants were all lower than that of the wild-type fusion protein GF, indicating that the four mutants had a better effect of activating two receptors at the same time. Among them, the mutant GFv5 had the lowest EC₅₀ value, indicating that it had the highest activity.

TABLE 1
Determination of GF protein activity
in HEK293-GLP1R/β-klotho/
CRE-Luciferase cells:

| | | | EC₅₀ |
|---|---|---|---|
| 93] | protein | 94] | (nM) |
| 95] | GF | 96] | 4.862 |
| 97] | GFv2 | 98] | 3.953 |
| 99] | GFv3 | 00] | 2.865 |
| 01] | GFv4 | 02] | 3.247 |
| 03] | GFv5 | 04] | 2.465 |

Example 4 Construction and Expression of GGF Fusion Protein with New Structure and Analysis of its Activity

Based on the four GF mutants, fusion proteins GGFv2, GGFv3, GGFv4, GGFv5 with new structures were constructed and expressed.

Specifically, the fusion proteins with new structures were represented by general formulas of G-L-G-L-Fc-L-Fv2 (GGFv2), G-L-G-L-Fc-L-Fv3 (GGFv3), G-L-G-L-Fc-L-Fv4 (GGFv4), G-L-G-L-Fc-L-Fv5 (GGFv5). Their amino acid sequences were as shown in SEQ ID NO:24-26 and 18, respectively. Their nucleotide sequences were as shown in SEQ ID NO:27-29 and 19.

The amino acid sequence of G-L-G-L-Fc-L-Fv2 was as shown in SEQ ID NO:24:

HGEGTFTSDVSSYLEEQAAKEFIAWLVKGGGGGGGSGGGGSGGGGSHGEG
TFTSDVSSYLEEQAAKEFIAWLVKGGGGGGGSGGGGSGGGGSAESKYGPP
CPPCPAPEAAGGPSVFLFPPKPKDTLMISRTPEVTCVVVDVSQEDPEVQF
NWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSC
SVMHEALHNHYTQKSLSLSLGGGGGSGGGGSGGGGSDSSPLLQFGGQVRQ
RYLYTDDAQQTEAHLEIREDGTVGGAADQSPESLLQLKALKPGVIQILGV
KTSRFLCQRPDGALYGSLHFDPEACSFRELLLEDGYNVYQSEAHGLPLHC
PGNKSPHRDPAPRGPCRFLPLPGLPPALPEPPGILAPQPPDVGSSDPLSM
VGGSQGRSPSYAS

The amino acid sequence of G-L-G-L-Fc-L-Fv3 was as shown in SEQ ID NO:25:

HGEGTFTSDVSSYLEEQAAKEFIAWLVKGGGGGGGSGGGGSGGGGSHGEG
TFTSDVSSYLEEQAAKEFIAWLVKGGGGGGGSGGGGSGGGGSAESKYGPP
CPPCPAPEAAGGPSVFLFPPKPKDTLMISRTPEVTCVVVDVSQEDPEVQF
NWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSC
SVMHEALHNHYTQKSLSLSLGGGGGSGGGGSGGGGSDSSPLLQFGGQVRQ
VYLYTDDAQQTEAHLEIREDGTVGGAADQSPESLLQLKALKPGVIQILGV
KTSRFLCQRPDGALYGSLHFDPEACSFRELLLEDGYNVYQSEAHGLPLHC
PGNKSPHRDPAPRGPCRFLPLPGLPPALPEPPGILAPQPPDVGSSDPLSM
VGGSQGRSPSYAS

The amino acid sequence of G-L-G-L-Fc-L-Fv4 was as shown in SEQ ID NO:26:

HGEGTFTSDVSSYLEEQAAKEFIAWLVKGGGGGGGSGGGGSGGGGSHGEG
TFTSDVSSYLEEQAAKEFIAWLVKGGGGGGGSGGGGSGGGGSAESKYGPP
CPPCPAPEAAGGPSVFLFPPKPKDTLMISRTPEVTCVVVDVSQEDPEVQF
NWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSC
SVMHEALHNHYTQKSLSLSLGGGGGSGGGGSGGGGSDSSPLLQFGGQVRQ
VYLYTDDAQQTEAHLEIREDGTVGGAADQSPESLLQLKALKPGVIQILGV
KTSRFLCQRPDGALYGSLHFDPEACSFRERLLEDGYNVYQSEAHGLPLHL
PGNKSPHRDPAPRGPARFLPLPGLPPALPEPPGILAPQPPDVGSSDPLSM
VGGSQGRSPSYES

The amino acid sequence of G-L-G-L-Fc-L-Fv5 was as shown in SEQ ID NO:18:

HGEGTFTSDVSSYLEEQAAKEFIAWLVKGGGGGGGSGGGGSGGGGSHGEG
TFTSDVSSYLEEQAAKEFIAWLVKGGGGGGGSGGGGSGGGGSAESKYGPP
CPPCPAPEAAGGPSVFLFPPKPKDTLMISRTPEVTCVVVDVSQEDPEVQF
NWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSC
SVMHEALHNHYTQKSLSLSLGGGGGSGGGGSGGGGSDSSPLLQFGGQVRQ
VYLYTDDAQQTEAHLEIREDGTVGGAADQSPESLLQLKALKPGVIQILGV
KTSRFLCQRPDGALYGSLHFDPEACSFRELLLEDGYNVYQSEAHGLPLHC
PGNKSPHRDPAPRGPCRFLPLPGLPPALPEPPGILAPQPPDVGSSDPLSM
VGGSQGRSPSYES

The nucleotide sequence of G-L-G-L-Fc-L-Fv2 was as shown in SEQ ID NO:27; the nucleotide sequence of G-L-G-L-Fc-L-Fv3 was as shown in SEQ ID NO:28; the nucleotide sequence of G-L-G-L-Fc-L-Fv4 was as shown in SEQ ID NO:29; the nucleotide sequence of G-L-G-L-Fc-L-Fv5 was as shown in SEQ ID NO:19.

The activation effects of four purified GGFv proteins on GLP1 receptor and FGF21 receptor were evaluated by using HEK293-GLP1R/β-klotho/CRE-Luciferase cells. See Example 3 for specific methods. As shown in FIG. 3A, the four GGFv proteins all had EC₅₀ values lower than that of the corresponding GFv protein, indicating that all the fusion proteins with new structures had improved activities. Among them, the activity of GGFv5 had been improved the most. As shown in FIG. 3B, GGFv5 and GFv5 both had EC₅₀ values lower than that of the drug dulaglutide (G, purchased from Eli Lilly and Company), and GGFv5 had the lowest EC₅₀ value, indicating that it had the highest activity.

Example 5 Verifying the Biological Activity of Bifunctional Protein in Hyperlipidemia Model Mice

24 low-density lipoprotein-deficient mice (Ld1r^{-/-} mice) (purchased from Jiangsu Jicui Yaokang Biotechnology Co., Ltd.), 4-8 weeks old, were fed with high-fat diet (containing 60% fat, purchased from Beijing Bai Ao Biotech Co., Ltd.) for 2 weeks, and then they became hyperlipidemia model mice. The mice were divided into 4 groups according to random body weights: control group (con, saline), G group (duraglutide), GFv5 group (GFv5 protein), GGFv5 group (GGFv5 protein), and each group had 6 mice. A dosage of 20 nmol/kg was administrated to each group twice a week by means of subcutaneous injection. The random body weights of mice were weighed and recorded every week. After 4 weeks of treatment, serum biochemical indicators were detected as follows: taking blood from eyeballs of mice; centrifuging at 3000 rpm for 10 minutes to separate serum; and sending samples to Beijing North Biomedical Technology Co., Ltd. for detecting triglyceride (TG), total cholesterol (TG), high-density lipids Protein (HDL), and low-density lipoprotein (LDL) indicators.

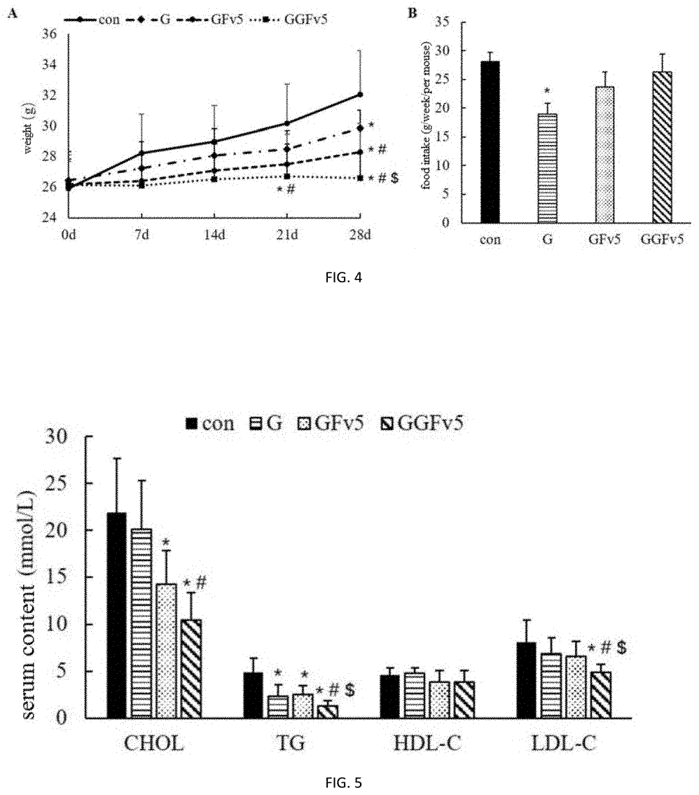

The results as shown in FIG. 4 indicated that, after treated with three drugs for four weeks, weights of mice in GGFv5 group, GFv5 group, and G group, were significantly lower than those in con group, particularly, GGFv5 group<GFv5 group<G group, and weights of mice in GFv5 group were also significantly lower than those in G group. Weights of mice in GGFv5 group were obviously different from those in the control group and G group just after treated only for 3 weeks, and were also obviously different from those in GFv5 group after 4 weeks. Moreover, for GGFv5 group, during the four weeks of observation, weights of the whole group of mice almost had no increase compared with those before the administration (weight gain rate was −0.48±2.23%). Observing the food intake of mice in each group during the treatment, it was found that, except that the food intake of mice in G group was significantly lower than those in con group, the food intake of mice in GGFv5 and GFv5 groups had no obvious difference from those in the control group. It demonstrated that difference in weights between mice in these two groups and the control group was not caused by the reduction in diet, however, the effect of drug G on weights of mice was probably related to the reduction in diet. Therefore, it showed that, in case of taking high-fat diet, by administrating GFv5 and GGFv5 drugs, the weight gain of mice can be controlled very well, and the effect of GGFv5 was especially better.

The results as shown in FIG. 5 showed that compared with con group mice, serum triglycerides (TG) of G group mice decreased significantly, cholesterol (CHOL) and TG of GFv5 group mice decreased significantly, and CHOL, TG and low density lipoprotein (LDL-C) level of GGFv5 group mice decreased significantly. In addition, CHOL, TG and LDL-C of GGFv5 group mice were significantly lower than those of G group mice, and TG and LDL-C of GGFv5 group mice were also obviously different from those of GFv5 group mice. It demonstrated that GFv5 and GGFv5 have good treatment effects on hyperlipidemia, and GGFv5 are even better.

The above description of the embodiments does not constitute any limitation to the scope of the present invention. Within the spirit of the present invention, various changes or modifications can be made by those skilled in the art, all of which fall within the scope of the appended claims.

What is claimed is:

1. A human fibroblast growth factor 21 variant having an amino acid sequence as shown in the following general formula I:
general formula I
DSSPLLQFGGQVRQX₁₅YLYTDDAQQTEAHLEIREDGTVGGAADQSPESL
LQLKALKPGVIQILGVKTSRFLCQRPDGALYGSLHFDPEACSFREX₉₄LL
EDGYNVYQSEAHGLPLHX₁₁₄PGNKSPHRDPAPRGPX₁₃₀RFLPLPGLPP
ALPEPPGILAPQPPDVGSSDPLSMVGGSQGRSPSYX₁₇₆S
wherein,
X₁₅ is Arg or Val,
X₉₄ is Leu or Arg,
X₁₁₄ is Leu or Cys,
X₁₃₀ is Ala or Cys, and
X₁₇₆ is Ala or Glu;
one and only one of X₉₄ and X₁₁₄ is Leu, and at most one of X₉₄ and X₁₁₄ is Ala;
and preferably, the amino acid sequence of the variant is as shown in any one of SEQ ID NO:1-4.

2. The human fibroblast growth factor 21 variant according to claim 1, wherein the human fibroblast growth factor 21 variant is prepared as a fusion protein, represented by the following general formula:
G-L-Fc-L-F, or
G-L-G-L-Fc-L-F
wherein,
F represents the human fibroblast growth factor 21 variant according to claim 1;
G represents a GLP-1 variant having an amino acid sequence shown in SEQ ID NO:5;
L represents a linker sequence; and
FC represents human or animal immunoglobulin and its subtypes and variants, human or animal albumin and its variants, or PEG.

3. The human fibroblast growth factor 21 variant according to claim 2, wherein the general formula of L is (GGGGS)n,
wherein
n is an integer from 0 to 5;
FC represents an IgG4 FC fragment, and preferably contains the amino acid sequence shown in SEQ ID NO:17;
the fusion protein further contains other antigens, functional amino acid sequences (histidine or GST tags) and/or signal peptide sequences;
and more preferably, the amino acid sequence of the fusion protein is as shown in any one of SEQ ID NO: 6-9, 18, or 24-26.

4. The human fibroblast growth factor 21 variant according to claim 1, wherein the human fibroblast growth factor 21 variant is encoded by a coding nucleotide sequence,
wherein, preferably, the coding nucleotide sequence of the fibroblast growth factor 21 variant is as shown in any one of SEQ ID NO:20-23, and
the coding nucleotide sequence of the fusion protein is as shown in any one of SEQ ID NO:10-13, 19, or 27-29.

5. The human fibroblast growth factor 21 variant according to claim 4, wherein the coding nucleotide sequence of the human fibroblast growth factor 21 is constructed into an expression construct.

6. The human fibroblast growth factor 21 variant according to claim 5, wherein the expression construct is a prokaryotic or eukaryotic expression construct;
preferably, the prokaryotic expression construct is a pET vector system; and the eukaryotic expression construct is a plasmid DNA vector, a recombinant viral vector, or a retroviral vector.

7. The human fibroblast growth factor 21 variant according to claim 5,
wherein, the expression construct is transfected into a host cells; when the expression construct is a prokaryotic expression construct, the host cell is a prokaryotic cell, preferably a bacterial cell; alternatively, when the expression construct is a eukaryotic expression construct, the host cell is a eukaryotic cell, preferably mammalian cell, more preferably CHO cell.

8. The human fibroblast growth factor 21 variant according to claim 1, wherein comprising the human fibroblast growth factor 21 variant is prepared in a pharmaceutical composition.

9. A method for preparing a fusion protein, comprising the step of cloning the coding nucleotide sequence of the human fibroblast growth factor 21 variant according to claim 1 into an expression vector,
wherein, preferably, the method comprises the steps as follows:
1) constructing the coding nucleotide sequence of the human fibroblast growth factor 21 variant;
2) constructing an expression vector containing the nucleotide sequence of step 1);
3) utilizing the expression vector of step 2) to transfect or transform a host cell and allow the nucleotide sequence to be expressed in the host cell;
and more preferably, in step 3), the host cell is a CHO-S cell.

10. A use of the human fibroblast growth factor 21 variant according to claim 1 in the preparation of drugs for obesity, hyperlipidemia, diabetes, and cardiovascular and cerebrovascular diseases.


SEQUENCE LISTING (29 sequences)

SEQ ID NO: 1 - protein, 177 aa, Artificial Sequence
  It is synthesized
DSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLSMVGGSQGRSPSYAS

SEQ ID NO: 2 - protein, 177 aa, Artificial Sequence
  It is synthesized
DSSPLLQFGGQVRQVYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLSMVGGSQGRSPSYAS

SEQ ID NO: 3 - protein, 177 aa, Artificial Sequence
  It is synthesized
DSSPLLQFGGQVRQVYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYES

SEQ ID NO: 4 - protein, 177 aa, Artificial Sequence
  It is synthesized
DSSPLLQFGGQVRQVYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLSMVGGSQGRSPSYES

SEQ ID NO: 5 - protein, 30 aa, Artificial Sequence
  It is synthesized
HGEGTFTSDVSSYLEEQAAKEFIAWLVKGG

SEQ ID NO: 6 - protein, 467 aa, Artificial Sequence
  It is synthesized
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLSMVGGSQGRSPSYAS

SEQ ID NO: 7 - protein, 467 aa, Artificial Sequence
  It is synthesized
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSDSSPLLQFGGQVRQVYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLSMVGGSQGRSPSYAS

SEQ ID NO: 8 - protein, 467 aa, Artificial Sequence
  It is synthesized
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSDSSPLLQFGGQVRQVYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYES

SEQ ID NO: 9 - protein, 467 aa, Artificial Sequence
  It is synthesized
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSDSSPLLQFGGQVRQVYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLSMVGGSQGRSPSYES

SEQ ID NO: 10 - DNA, 1404 bp, Artificial Sequence
  It is synthesized
cacggcgagggcaccttcacctccgacgtgtcctcctatctcgaggagcaggccgccaaggaattcatcgcctggctggtgaagggcggcggcggtggtggtggctccggaggcggcggctctggtggcggtggcagcgctgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtggcggaggcggaagcggaggcggaggaagcggcggtggcggcagcgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcactgcccagggaacaagtccccacaccgggaccctgcaccccgaggaccatgccgcttcctgccactaccaggcctgccccccgcactcccggagccacccggaatcctggccccccagccccccgatgtgggctcctcggaccctctgagcatggtgggaggctcccagggccgaagccccagctacgcttcctga

SEQ ID NO: 11 - DNA, 1404 bp, Artificial Sequence
  It is synthesized
cacggcgagggcaccttcacctccgacgtgtcctcctatctcgaggagcaggccgccaaggaattcatcgcctggctggtgaagggcggcggcggtggtggtggctccggaggcggcggctctggtggcggtggcagcgctgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtggcggaggcggaagcggaggcggaggaagcggcggtggcggcagcgactccagtcctctcctgcaattcgggggccaagtccggcaggtgtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcactgcccagggaacaagtccccacaccgggaccctgcaccccgaggaccatgccgcttcctgccactaccaggcctgccccccgcactcccggagccacccggaatcctggccccccagccccccgatgtgggctcctcggaccctctgagcatggtgggaggctcccagggccgaagccccagctacgcttcctga

SEQ ID NO: 12 - DNA, 1404 bp, Artificial Sequence
  It is synthesized
cacggcgagggcaccttcacctccgacgtgtcctcctatctcgaggagcaggccgccaaggaattcatcgcctggctggtgaagggcggcggcggtggtggtggctccggaggcggcggctctggtggcggtggcagcgctgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtggcggaggcggaagcggaggcggaggaagcggcggtggcggcagcgactccagtcctctcctgcaattcgggggccaagtccggcaggtgtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagcggcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaggctcccagggccgaagccccagctacgagtcctga

SEQ ID NO: 13 - DNA, 1404 bp, Artificial Sequence
  It is synthesized
cacggcgagggcaccttcacctccgacgtgtcctcctatctcgaggagcaggccgccaaggaattcatcgcctggctggtgaagggcggcggcggtggtggtggctccggaggcggcggctctggtggcggtggcagcgctgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtggcggaggcggaagcggaggcggaggaagcggcggtggcggcagcgactccagtcctctcctgcaattcgggggccaagtccggcaggtgtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcactgcccagggaacaagtccccacaccgggaccctgcaccccgaggaccatgccgcttcctgccactaccaggcctgccccccgcactcccggagccacccggaatcctggccccccagccccccgatgtgggctcctcggaccctctgagcatggtgggaggctcccagggccgaagccccagctacgagtcctga

SEQ ID NO: 14 - protein, 471 aa, Artificial Sequence
  It is synthesized
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 15 - DNA, 1416 bp, Artificial Sequence
  It is synthesized
cacggcgagggcaccttcacctccgacgtgtcctcctatctcgaggagcaggccgccaaggaattcatcgcctggctggtgaagggcggcggcggtggtggtggctccggaggcggcggctctggtggcggtggcagcgctgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtggcggaggcggaagcggaggcggaggaagcggcggtggcggcagccaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctga

SEQ ID NO: 16 - DNA, 72 bp, Artificial Sequence
  It is synthesized
atgccgtcttctgtctcgtggggcatcctcctgctggcaggcctgtgctgcctggtccctgtctccctggct

SEQ ID NO: 17 - protein, 229 aa, Artificial Sequence
  It is synthesized
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 513 aa, Artificial Sequence
  It is synthesized
HGEGTFTSDVSSYLEEQAAKEFIAWLVKGGGGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSDSSPLLQFGGQVRQVYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLSMVGGSQGRSPSYES

SEQ ID NO: 19 - DNA, 1542 bp, Artificial Sequence
  It is synthesized
catggcgaagggacctttaccagtgatgtaagttcttatttggaagagcaagctgccaaggaattcattgcttggctggtgaaaggcggcggaggcggaggcggaagcggaggcggaggaagcggcggtggcggcagccacggcgagggcaccttcacctccgacgtgtcctcctatctcgaggagcaggccgccaaggaattcatcgcctggctggtgaagggcggcggcggtggtggtggctccggaggcggcggctctggtggcggtggcagcgctgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtggcggaggcggaagcggaggcggaggaagcggcggtggcggcagcgactccagtcctctcctgcaattcgggggccaagtccggcaggtgtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcactgcccagggaacaagtccccacaccgggaccctgcaccccgaggaccatgccgcttcctgccactaccaggcctgccccccgcactcccggagccacccggaatcctggccccccagccccccgatgtgggctcctcggaccctctgagcatggtgggaggctcccagggccgaagccccagctacgagtcctga

SEQ ID NO: 20 - DNA, 534 bp, Artificial Sequence
  It is synthesized
gactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcactgcccagggaacaagtccccacaccgggaccctgcaccccgaggaccatgccgcttcctgccactaccaggcctgccccccgcactcccggagccacccggaatcctggccccccagccccccgatgtgggctcctcggaccctctgagcatggtgggaggctcccagggccgaagccccagctacgcttcctga

SEQ ID NO: 21 - DNA, 534 bp, Artificial Sequence
  It is synthesized
gactccagtcctctcctgcaattcgggggccaagtccggcaggtgtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcactgcccagggaacaagtccccacaccgggaccctgcaccccgaggaccatgccgcttcctgccactaccaggcctgccccccgcactcccggagccacccggaatcctggccccccagccccccgatgtgggctcctcggaccctctgagcatggtgggaggctcccagggccgaagccccagctacgcttcctga

SEQ ID NO: 22 - DNA, 534 bp, Artificial Sequence
  It is synthesized
gactccagtcctctcctgcaattcgggggccaagtccggcaggtgtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagcggcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaggctcccagggccgaagccccagctacgagtcctga

SEQ ID NO: 23 - DNA, 534 bp, Artificial Sequence
  It is synthesized
gactccagtcctctcctgcaattcgggggccaagtccggcaggtgtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcactgcccagggaacaagtccccacaccgggaccctgcaccccgaggaccatgccgcttcctgccactaccaggcctgccccccgcactcccggagccacccggaatcctggccccccagccccccgatgtgggctcctcggaccctctgagcatggtgggaggctcccagggccgaagccccagctacgagtcctga

SEQ ID NO: 24 - protein, 513 aa, Artificial Sequence
  It is synthesized
HGEGTFTSDVSSYLEEQAAKEFIAWLVKGGGGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLSMVGGSQGRSPSYAS

SEQ ID NO: 25 - protein, 512 aa, Artificial Sequence
  It is synthesized
HGEGTFTSDVSSYLEEQAAKEFIAWLVKGGGGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSDSSPLLQFGGQVRQVYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHCPGNKSPHRDPAPRGPCRFLPLPGLPPALPEPPGILAPQPPDVGSSDPLSMVGGSQGRSPSYA

SEQ ID NO: 26 - protein, 513 aa, Artificial Sequence
  It is synthesized
HGEGTFTSDVSSYLEEQAAKEFIAWLVKGGGGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSDSSPLLQFGGQVRQVYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPALPEPPGILAPQPPDVGSSDPLSMVGGSQGRSPSYES

SEQ ID NO: 27 - DNA, 1542 bp, Artificial Sequence
  It is synthesized
catggcgaagggacctttaccagtgatgtaagttcttatttggaagagcaagctgccaaggaattcattgcttggctggtgaaaggcggcggaggcggaggcggaagcggaggcggaggaagcggcggtggcggcagccacggcgagggcaccttcacctccgacgtgtcctcctatctcgaggagcaggccgccaaggaattcatcgcctggctggtgaagggcggcggcggtggtggtggctccggaggcggcggctctggtggcggtggcagcgctgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtggcggaggcggaagcggaggcggaggaagcggcggtggcggcagcgactccagtcctctcctgcaattcgggggccaagtccggcaggtgtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagcggcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaggctcccagggccgaagccccagctacgagtcctga

SEQ ID NO: 28 - DNA, 1542 bp, Artificial Sequence
  It is synthesized
catggcgaagggacctttaccagtgatgtaagttcttatttggaagagcaagctgccaaggaattcattgcttggctggtgaaaggcggcggaggcggaggcggaagcggaggcggaggaagcggcggtggcggcagccacggcgagggcaccttcacctccgacgtgtcctcctatctcgaggagcaggccgccaaggaattcatcgcctggctggtgaagggcggcggcggtggtggtggctccggaggcggcggctctggtggcggtggcagcgctgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtggcggaggcggaagcggaggcggaggaagcggcggtggcggcagcgactccagtcctctcctgcaattcgggggccaagtccggcaggtgtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcactgcccagggaacaagtccccacaccgggaccctgcaccccgaggaccatgccgcttcctgccactaccaggcctgccccccgcactcccggagccacccggaatcctggccccccagccccccgatgtgggctcctcggaccctctgagcatggtgggaggctcccagggccgaagccccagctacgcttcctga

SEQ ID NO: 29 - DNA, 1542 bp, Artificial Sequence; identical to SEQ ID NO: 27
  It is synthesized